(19) United States
(12) Patent Application Publication
(10) Pub. No.: US 2004/0022675 A1
(43) Pub. Date: Feb. 5, 2004

(54) Humidifying method and apparatus using silver ionized water

(76) Inventor: Jung-O An, Seoul (KR)

Correspondence Address: GREENBLUM & BERNSTEIN, P.L.C., 1950 ROLAND CLARKE PLACE, RESTON, VA, 20191, US

(21) Appl. No.: 10/416,539

(22) PCT Filed: Nov. 16, 2001
(86) PCT No.: PCT/KR01/01961

(30) Foreign Application Priority Data
Nov. 29, 2000 (KR) 2000-33479UM
Mar. 20, 2001 (KR) 2001-14224
Oct. 27, 2001 (KR) 2001-66520

(51) Int. Cl. A61L 9/00

(52) U.S. Cl. 422/29

(57) ABSTRACT

The present invention relates to a humidifying method and apparatus using silver ionized water. In the present invention, tap water, distilled water or purified water is produced to a silver solution and a silver ionized water is atomized by ultraviolet vibrator. Sterilization effects in the air are enhanced and infections from a harmful virus in the respiratory organs are prevented.

### TECHNICAL FIELD

The present invention relates to a humidifying method and apparatus using silver ionized water. More particularly, the method comprises producing the silver ionized water from general water, vaporizing the produced silver ionized water by an ultrasonic wave oscillator, heating means or centrifugal means, and spraying the vaporized silver ionized water into the air, thereby enhancing sterilization effects and preventing respiratory ailments caused by harmful bacteria or virus.

### BACKGROUND ART

Generally, in the winter when the rainfall is insufficient and the humidity falls greatly due to freezing of moisture by cold, the surrounding air is very dry, moreover the indoor air is exceedingly dry by the heat evolved from a heater used in home or office. If the dry indoor is lasted, such a condition is detrimental to human health.

Accordingly, to get rid of such a problem, a humidifier for creating the moisture artificially in the dry air is used. The humidifier can be classified into two large groups based on how to generate the moisture. One is a heating type humidifier in which the water in a water reservoir is heated up to the predetermined temperature, thereby the vapor created by the heating is sprayed naturally into the air. The other is an ultrasonic wave type humidifier in which the water in a water reservoir is minute by oscillation of ultrasonic wave, thereby the minute water particles are sprayed into the air.

However, it is very difficult to regulate the amount of humidification in the heating type humidifier since it humidifies the air simply using the vapor generated by heating the water. Consequently, the consumer has preferred the ultrasonic wave type humidifier capable of suitably regulating the amount of humidification.

The conventional ultrasonic wave humidifier comprises a body having a water reservoir disposed in a upper part thereof to contain water and a humidifying chamber, a water tank mounted to said water reservoir for storing and supplying the water, an oscillator assembly installed in said humidifying chamber for generating the vapor of fine particles by vibration of tie water and a vapor tube for inducing the vapor into the indoor.

The ultrasonic wave humidifier further comprises a water inlet formed on a lower end of said water tank for intermittently discharging the water to said water reservoir, a vapor outlet formed on a upper part of said vapor tube for controlling a spraying direction of the vapor and a fan installed in an inner space of said body for smoothly spraying the vapor into the indoor through said vapor tube.

The oscillator assembly comprises a oscillator for generating an ultrasonic wave by oscillation thereof to convert the water into the vapor, a rubber packing having a cylindrical hollow therein in which said oscillator is inserted with inclined shape in said cylindrical hollow so that it is operable to be oscillated, a oscillator cover attached in screw form to a bottom surface of said humidifying chamber and having an insertion groove being concaved toward a bottom thereof to allow said rubber packing to be inserted therein, and a case installed in a lower part of said oscillator cover and having electromagnetic control means for oscillating said oscillator therein.

As described above, the water stored in said water tank intermittently passes to said humidifying chamber via said water reservoir through said water inlet. The oscillator is oscillated in said rubber packing by said electromagnetic control means and creates ultrasonic waves, and generates a column of water. Consequently, the water contained in said humidifying chamber is vaporized in the form of minute particles and the produced vapor is sprayed into the indoor through said vapor outlet along said vapor tube by said fan.

Recently, considering that it is very harmful to a human body, especially a patient or infant when the water is not sterilized and sprayed into the indoor, the humidifier is provided with a sterilizing chamber having a heater or with a heater installed in a humidifying chamber so that by heating the water and relaxing water molecules, the vaporization efficiency according to oscillation of an oscillator is enhanced. However, in such a case, the oscillator is heated over a Curie point of 140-150° C. by humidification of heated water and its magnetic properties are changed, therefore its efficiency and lifetime are greatly deteriorated.

### DISCLOSURE OF INVENTION

To overcome the problems described above, preferred embodiments of the present invention provide a humidifying method and apparatus using silver ionized water, wherein the method comprises producing the silver ionized water from general water, vaporizing the produced silver ionized water by ultrasonic waves, and spraying the vaporized silver ionized water into the air, thereby enhancing sterilization effects and preventing respiratory ailments caused by harmful bacteria or virus.

It is other object of the present invention to provide a humidifying method and apparatus using silver ionized water needless to sterilize inner parts of the humidifier separately.

It is another object of the present invention to provide a humidifying method and apparatus using silver ionized water obtained from distilled water.

It is another object of the present invention to provide a humidifying method and apparatus using silver ionized water obtained from general water.

In order to accomplish these objects, there is provided a humidifying method using silver ionized water, wherein the method comprises producing said silver ionized water by electrolysis of a plurality of silver rods or silver plates immersed in general water, distilled water or purified water and connected to a DC or AC electric power source; vaporizing said silver ionized water produced in said step by an ultrasonic wave oscillator, heating means or centrifugal means; and spraying said vaporized silver ionized water into air, whereby said air is humidified and sterilized.

The purified water can be obtained from purification of said general water in an ion exchange resin system disposed in a lower end of water storing and supplying means and the distilled water can be obtained from distillation of said general water by heating and cooling.

In accordance with an aspect of the present invention, there is provided a humidifying method in which said distilled water is produced by heating of said general water in distilled water generating means; said silver ionized water is produced using said distilled water in silver ionized water generating means; said silver ionized water is vaporized by oscillation of said ultrasonic wave oscillator; thereby humidification by said silver ionized water is continually performed.

In accordance with other aspect of the present invention, there is provided a humidifying method in which said silver ionized water is rapidly produced using said general water by method for providing said plurality of silver rods or silver plates with a DC electric power source of high voltage; or providing said plurality of silver rods or silver plates with a DC or AC electric power source of high voltage after heating said plurality of silver rods or silver plates by a heater connected thereto.

The silver rod or silver plate used for production of the silver ionized water in the to present invention has a purity of at least 99.99%, and a size of silver particle electrolyzed is 0.005-0.015 μm. The nano-size no more than 0.015 μm maximizes sterilization effects and other properties of silver.

A humidifying apparatus using silver ionized water comprises a body; a cover; water storing, and supplying means; silver ionized water generating means; humidification generating means comprising vaporizing means having an ultrasonic wave oscillator, heating means or centrifugal means, and spraying means having a ventilating fan; and control means.

In accordance with another aspect of the present invention, there is provided a humidifying apparatus, further comprising an ion exchange resin system for purifying general water disposed in a lower end of said water storing and supplying means; wherein said silver ionized water generating means are mounted in a lower end of said ion exchange resin system and comprise a plurality of silver rods or silver plates therein, said plurality of silver rods or silver plates being connected to an electric power source to be electrolyzed in water purified from said ion exchange resin system; wherein said vaporizing means have said ultrasonic wave oscillator, whereby said silver ionized water is vaporized by said ultrasonic wave oscillator and sprayed into air.

In accordance with further aspect of the present invention, there is provided a humidifying apparatus, further comprising distilled water generating means which comprise a water reservoir, heat exchanging and vapor cooling means, a cooling plate, a cooling fan, a heater for heating said general water, a water level sensor for controlling a water level constantly, a water tube and a vapor tube; wherein said silver ionized water generating means have a plurality of silver rods or silver plates therein, said plurality of silver rods or silver plates being connected to an electric power source to be electrolyzed in water distilled from said distilled water generating means, further comprising a microcomputer for providing said plurality of silver rods or silver plates with electric power source cross-alternatively; wherein said humidification generating means further comprise a filter for removing any impurities from said silver ionized water, whereby said silver ionized water is vaporized by said ultrasonic wave oscillator and sprayed into air by said a ventilating fan.

In accordance with another aspect of the present invention, there is provided a humidifying apparatus, further comprising silver rod or silver plate fixing means having a plurality of silver rods or silver plates mounted to said water storing and supplying means; electric power source supplying means comprising electric contacting means electrically contacted with said plurality of silver rods or silver plates and a spring mounted to said body; and a circuit breaker for breaking an electric power source when said cover opens.

In accordance with further aspect of the present invention, there is provided a humidifying apparatus in which said control means comprises a flicker lamp mounted on a front panel of said body for displaying a electrolysis process in order while said control means provide said plurality of silver rods or silver plates with a DC or AC electric power source through said electric contacting means and silver ionization is performed by electrolysis of said plurality of silver rods or silver plates; a sensor for preventing an electric leakage or short circuit caused by moisture; and a circuit breaker comprising a lead switch mounted on said body and a magnetic body mounted to said cover; whereby all parts or processes are controlled by said control means.

In accordance with another aspect of the present invention, there is provided a humidifying apparatus, further comprising a separate space formed on a central part of said body, said plurality of silver rods or silver plates being mounted in said separate space instead of being mounted in said water storing and supplying means; wherein said plurality of silver rods or silver plates are provided with an electric power source of high voltage cross-alternatively or together with heat in order to rapidly produce said silver ionized water; whereby said silver ionized water is vaporized by said ultrasonic wave oscillator and sprayed into air by said a ventilating fan.

### BRIEF DESCRIPTION OF DRAWINGS

For a more complete under supporting of the present invention and the advantages thereof, reference is now made to the following descriptions taken in conjunction with the accompanying drawings, in which like reference numerals denote like parts, and in which:

FIG. 1 is a disassembled perspective view illustrating a humidifying apparatus using silver ionized water according to a preferable embodiment of the present invention;

FIG. 2 is a cross-sectional view of FIG. 1;

FIG. 3 is a partially incised perspective view illustrating a humidifying apparatus using silver ionized water according to other embodiment of the present invention;

FIG. 4 is a partially incised cross-sectional view of FIG. 3;

FIG. 5 is a cross-sectional view illustrating distilled water generating means in FIG. 3;

FIG. 6 is a cross-sectional view illustrating silver ionized water generating means in FIG. 3;

FIG. 7 is an external view illustrating a conventional humidifier;

FIG. 8 is a plan view illustrating a humidifying apparatus using silver ionized water according to another embodiment of the present invention;

FIG. 9 is a cross-sectional view illustrating water storing and supplying means mounted to a body in FIG. 8;

FIG. 10 is a cross-sectional view illustrating electric power source supplying means and electric contacting means between a body and water storing and supplying means;

FIG. 11 is a cross-sectional view illustrating a configuration for breaking an electric power source when a cover opens;

FIG. 12 is a cross-sectional view illustrating electric contacting means mounted to a body according to other embodiment of FIG. 8;

FIG. 13 is a cross-sectional view illustrating other embodiment of FIG. 10;

FIG. 14 is a cross-sectional view illustrating an electric power source breaker according to other embodiment of FIG. 11;

FIG. 15 is a cross-sectional vie illustrating a space formed inside a body according to another embodiment of the present invention.

### BEST MODE FOR CARRYING OUT THE INVENTION

Reference will now be made in detail to preferred embodiments of the present invention, example of which is illustrated in the accompanying drawings. Like reference numerals denote like parts.

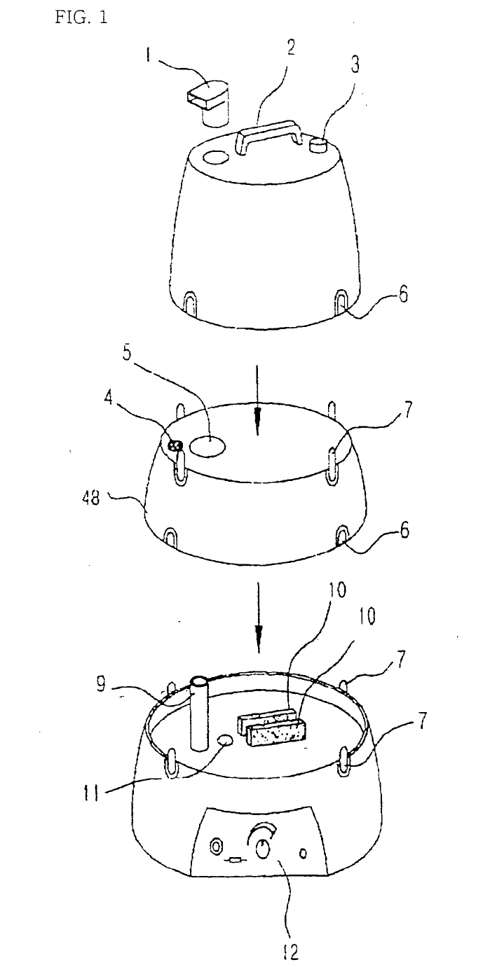

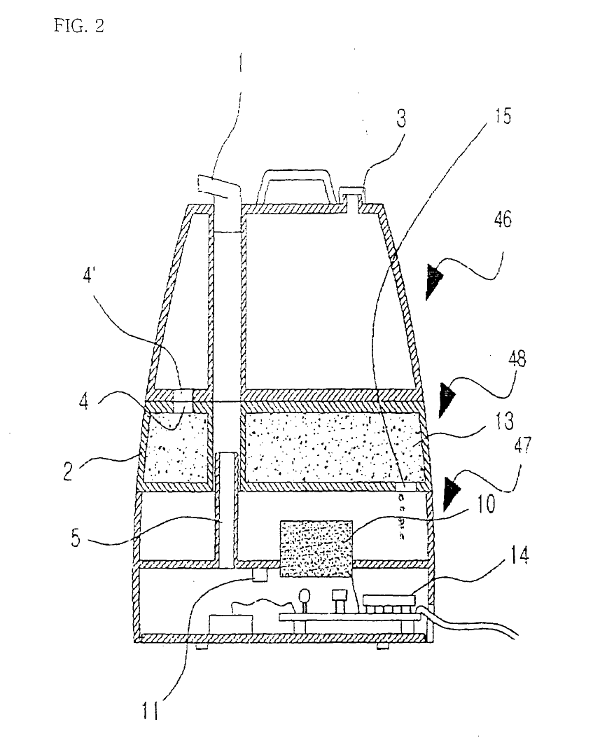

As shown in FIG. 1 and FIG. 2, a humidifying apparatus of the present invention comprises an ion exchange resin system 48 disposed in a lower end of said water storing and supplying means 46. A plurality of silver rods or silver plates 10 are connected to an electric power source and are electrolyzed in water purified from said ion exchange resin system 48, and then silver ionized water is generated.

In more detail, the humidifying apparatus comprises water stoning and supplying means 46; an ion exchange resin system 48 for removing a metal and non-metal disposed in the lower end of said water storing and supplying means 46; silver ionized water generating, means 47 mounted in a lower end of said ion exchange resin system 48 and having a plurality of silver rods or silver plates 10 therein, wherein said plurality of silver rods or silver plates 10 are connected to an electric power source to be electrolyzed in water purified from said ion exchange resin system 48; and an ultrasonic wave oscillator 11 for vaporizing the silver ionized water.

The water storing and supplying means 46 comprise a vapor outlet 1 vertically piercing a upper part thereof, a handle 2, a water stopper 3, and a water outlet 4′ communicating with said ion exchange resin system 48.

The ion exchange resin system 48 comprises a vapor tube 5 formed on an end surface thereof, a water inlet 4, a water outlet 15 formed on a bottom surface thereof, and an ion exchange resin 13 for removing any impurities such as a metal or non-metal in order to smoothly, generate the silver ionized water.

The silver ionized water generating means 47 are mounted in the lower end of said ion exchange resin system 48 and have a plurality of silver rods or silver plates 10 therein Said plurality of silver rods or silver plates 10 are connected to the electric power source and are electrolyzed in water purified from said ion exchange resin system 48. Then silver ionized water is generated by such an electrolysis process and vaporized by said ultrasonic wave oscillator 11, and sprayed into the air.

To be electrolyzed equally, said plurality of silver rods or silver plates 10 are cross-alternatively provided with a DC or AC electric power source. These operations are controlled by a control panel 12 and a microcomputer 14.

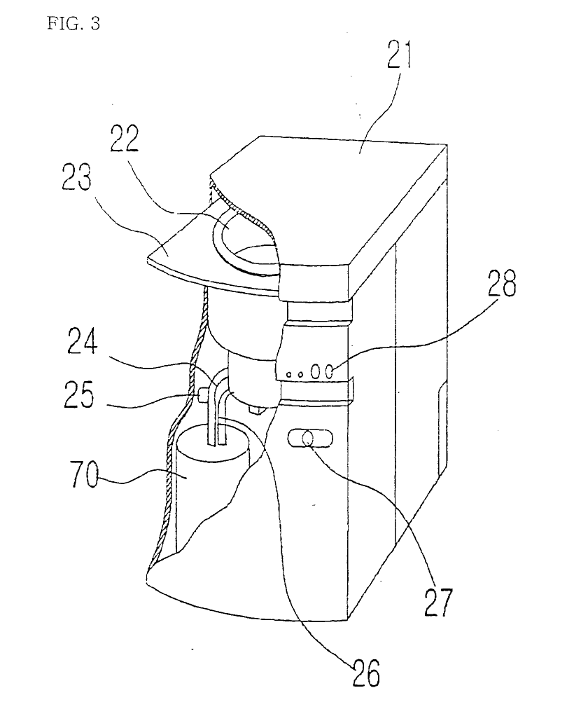

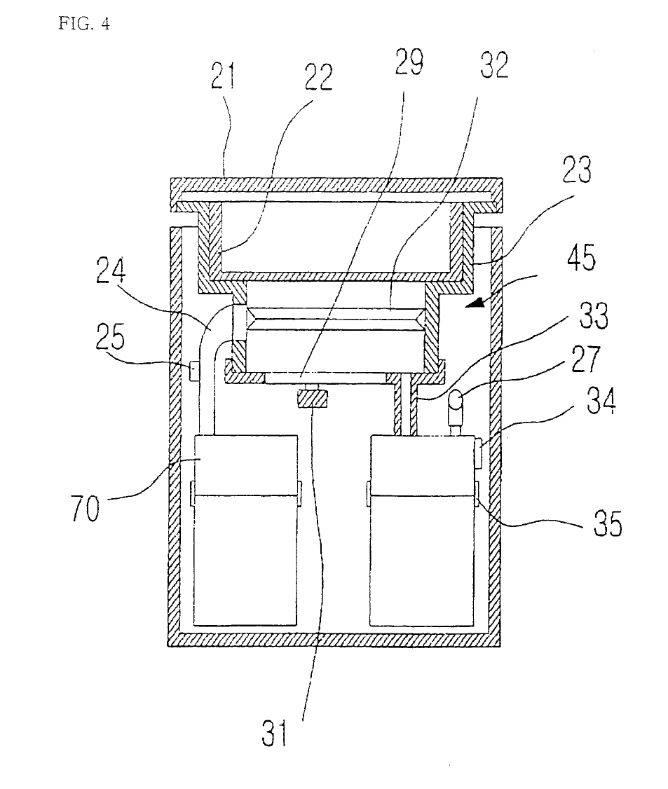

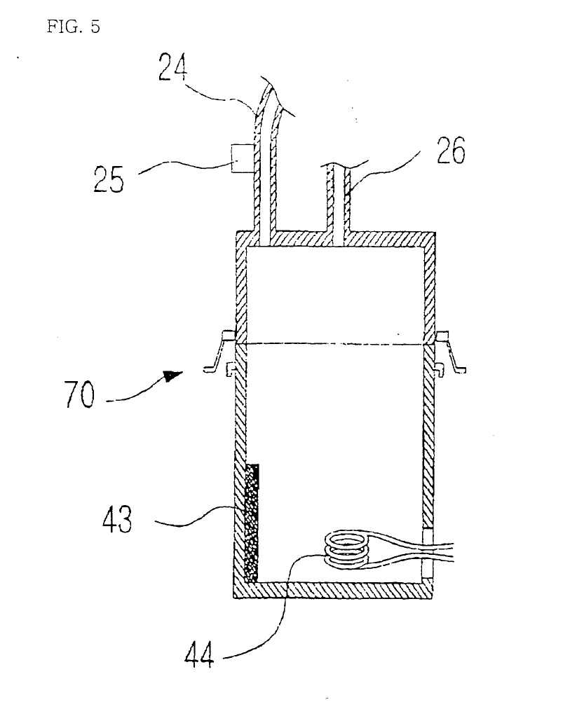

Referring to FIG. 3 to FIG. 6, other humidifying apparatus of the present invention comprises distilled water generating means 70 for producing distilled water by heating general water; silver ionized water generating means 50 for creating silver ionized water using the distilled water obtain in said distilled water generating means 70; and humidification generating means 60 for vaporizing and spraying the silver ionized water, thereby humidification is continually performed.

The distilled water generating means 70 comprise a water tank 22, a water supply tube 24 connected to said water tank 22, opening and shutting means 25 mounted on said water supply tube 24, a cooling plate 32 interposed between said water tank 22 and a vapor storing chamber, heat exchanging and vapor cooling means 45 having a plurality of radiating plates mounted thereto for cooling the vapor, a heater 44 for heating the water, and a water level sensor 43 for measuring a constant water level. The opening and shutting means 25 open and shut by sensing when said heater 44 is overheated.

The heat transfer between the water entering said water supply tube 24 from said water tank 22 and the vapor going out from a vapor supply tube 26 is made on said cooling plate 32.

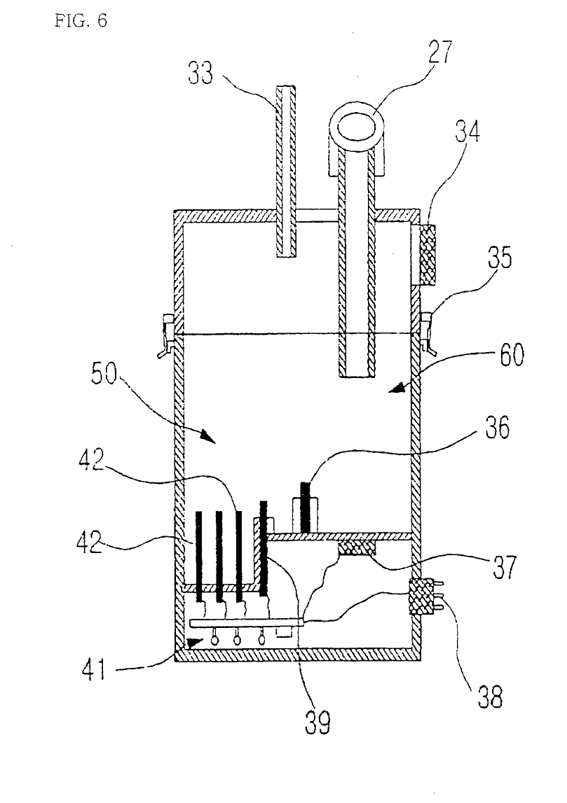

Referring to FIG. 6, the silver ionized water generating means 50 comprise a plurality of silver rods or silver plates 42 therein, and a circuit controller 41 including a microcomputer and electrically connected to said plurality of silver rods or silver plates 42 so that said plurality of silver rods or silver plates 42 are cross-alternatively provided with an electric power source.

As shown in FIG. 6, the humidification generating means 60 comprise a filter 36 for removing any impurities from said silver ionized water, an ultrasonic wave oscillator 37 for vaporizing the silver ionized water, and a ventilating fan 34 for spraying the vaporized silver ionized water.

The humidifying methods of the above humidifying apparatus are described as below in detail.

First, an electric power source is applied to control means and provided with said heater 44. The water is supplied to said water tank 92 and vaporized by said heater 44 to be vapor. The vapor passes through said vapor supply tube 26 and is cooled in said cooling plate 32 to be distilled water. The heat efficiency is high since heat transfer between the water and the vapor is made on said cooling plate 32. The cooling plate 32 is made of titanium or stainless materials by insertion process on forming plastic.

In other embodiment of the present invention, said heat exchanging and vapor cooling means 45 can be formed inside a plate having a plurality of holes. Also, to cool the vapor, a cooling fan can be further installed outside vapor outlet.

The distilled water is supplied to said silver ionized water generating means 50 in which the silver ionized water is produced by electrolysis of said plurality of silver rods or silver plates 42. Said plurality of silver rods or silver plates 10 are connected to the electric power source and cross-alternatively provided with a DC or AC electric power source by said a circuit controller 41 including a microcomputer.

The silver ionized water is filtered and vaporized by said ultrasonic wave oscillator 37 in said humidification generating means 60. The ultrasonic wave oscillator 37 creates ultrasonic waves, and generates a column of water. Consequently, the silver ionized water is vaporized in the form of minute particles. The ultrasonic wave oscillator 37 is cooled in said heat exchange and vapor cooling means 45.

The vaporized silver ionized water is sprayed into the indoor through a vapor outlet 27 along a vapor tube by said ventilating fan 34. The control means regulate the amount of humidification according to the temperature and humidity of the indoor.

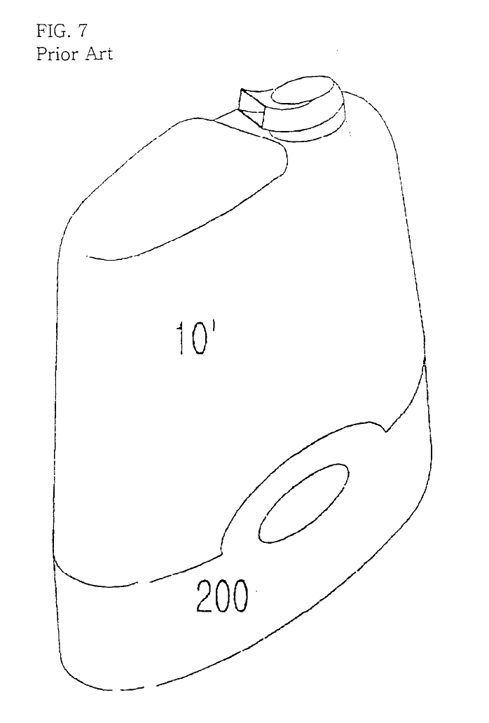

To help the understanding of the present invention, a conventional humidifier is illustrated in FIG. 7.

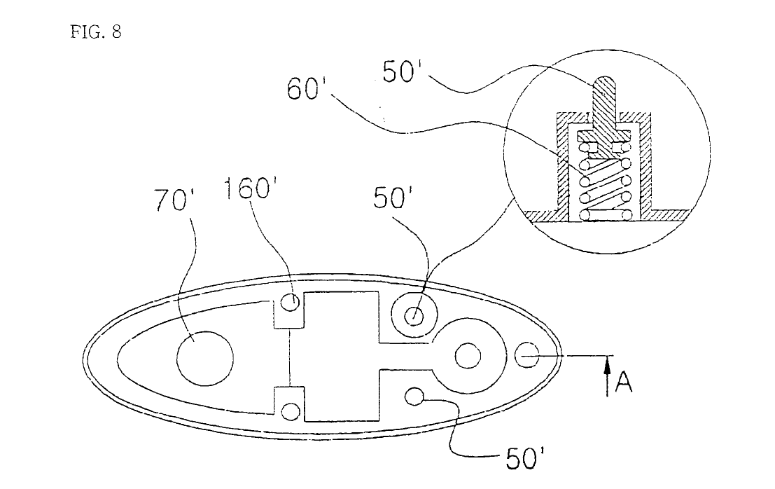

Referring to FIG. 8 to FIG. 11, another humidifying apparatus of the present invention comprises water storing and supplying means 190, a plurality of silver rods or silver plates 90′ mounted to said water storing and supplying means 190, electric contacting means 50′ of a body 200, and an oscillator 70. The plurality of silver rods or silver plates 90′ are electrically connected to an electric power source and have a purity of at least 99.99%.

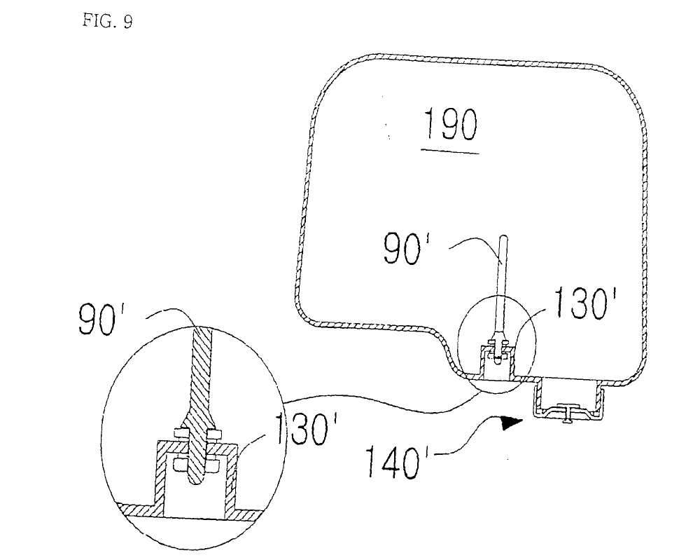

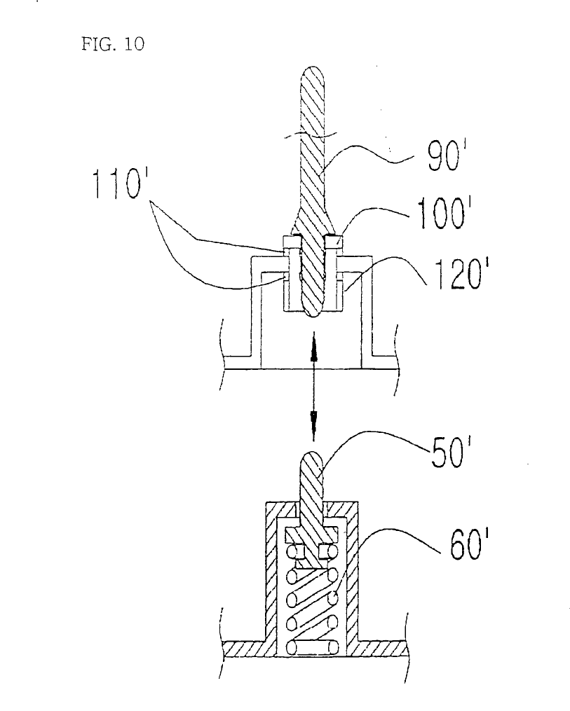

As shown in FIG. 9 and FIG. 10, the water storing and supplying means 190 have a contact groove 130′ formed on both sides thereof, a bolt 100′, a nut 120′ and a seal packing 110′. The plurality of silver rods or silver plates 90′ are inserted with said a contact groove 130′ using said a bolt 100′, a nut 120′ and a seal packing 110′.

As shown in FIG. 10, the plurality of silver rods or silver plates 90′ are electrically connected to push contacting means 50′ of said body 200 to be electrolyzed. The push contacting means 50′ are connected to a spring 60′ to be smoothly contacted.

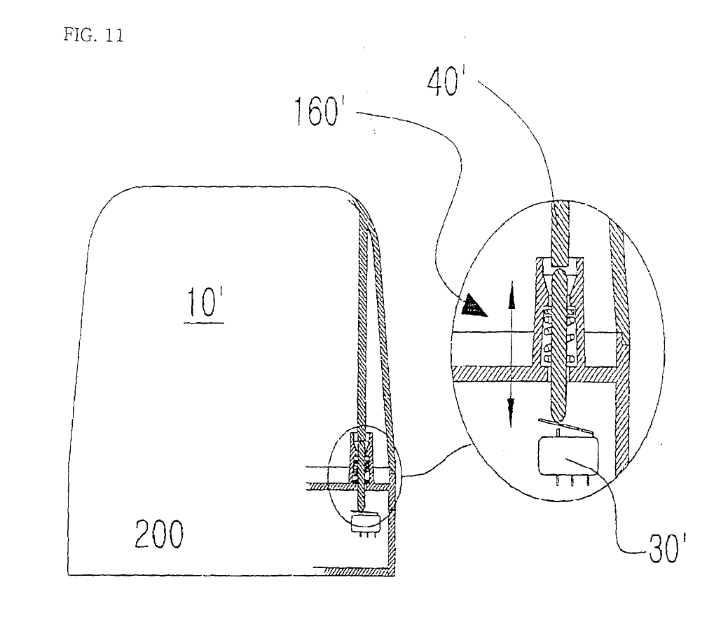

As shown in FIG. 11, a circuit breaker 160′ comprises a push switch 30′ mounted on said body and a contact rod 40′ mounted to said cover 10′. When said cover opens, said push switch 30′ being pushed by said contact rod 40′ is off, thereby the electric power source is broken.

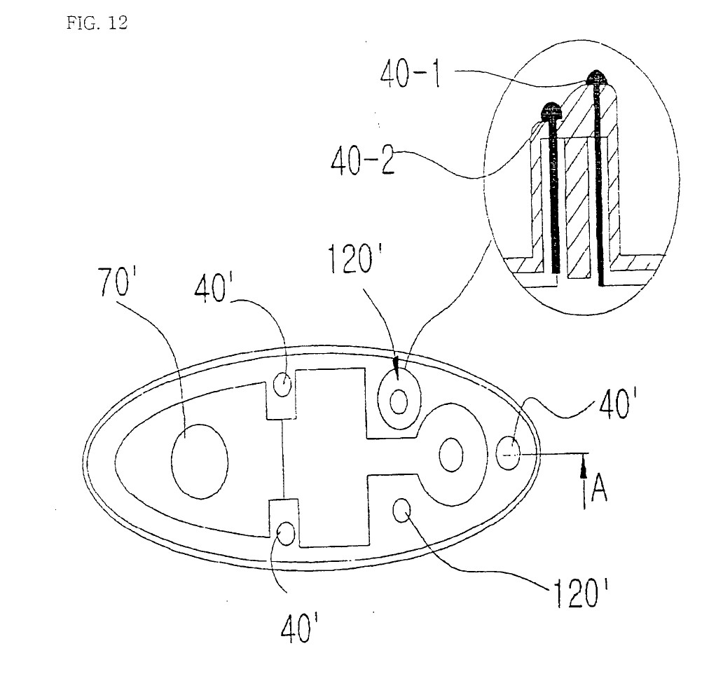

FIG. 12 shows another embodiment of the present invention, wherein electric power source supplying means contact with water storing and supplying means, and comprise a extruding contact rod 40-1 and a sensor 40-2 formed in a predetermined position for sensing an electric leakage caused by moisture.

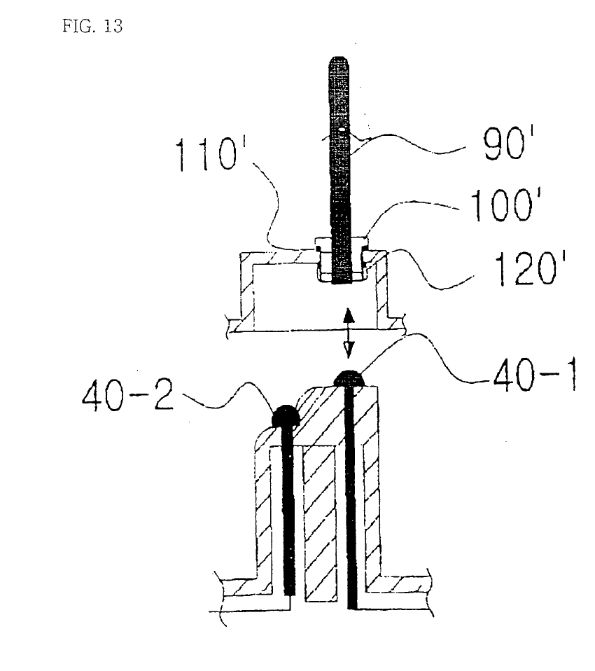

FIG. 13 shows a connection between electric power source supplying means and water storing and supplying means, wherein said electric power source supplying means comprise a extruding contact rod 40-1 and a sensor 40-2 for sensing an electric leakage in order to contact with a plurality of silver rods or silver plates mounted to said water storing and supplying means.

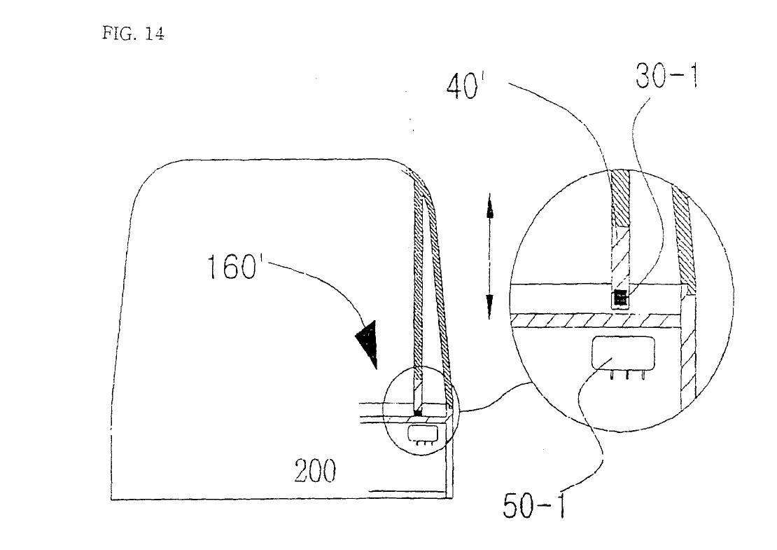

FIG. 14 shows another embodiment of the present invention, wherein a circuit breaker 160′ comprises a lead switch 50-1 mounted on a body and a magnetic body 30-1 mounted to a cover, thereby an electric power source is broken when said cover opens.

The humidifying methods of the above humidifying apparatus are described as below in detail.

When an electric power source and water are supplied to said water storing and supplying means 190, the electric power source is applied through said electric contacting means 50, 40-1 to said plurality of silver rods or silver plates 60′, 90′ mounted to said water storing and supplying means 190. Then, the plurality of silver rods or silver plates 60′, 90′ are electrolyzed to be silver ionized water. The produced silver ionized water is discharged along a cover 140′ disposed in a bottom end of said water storing and supplying means 190, and vaporized by said ultrasonic wave oscillator 70′ and sprayed into the air.

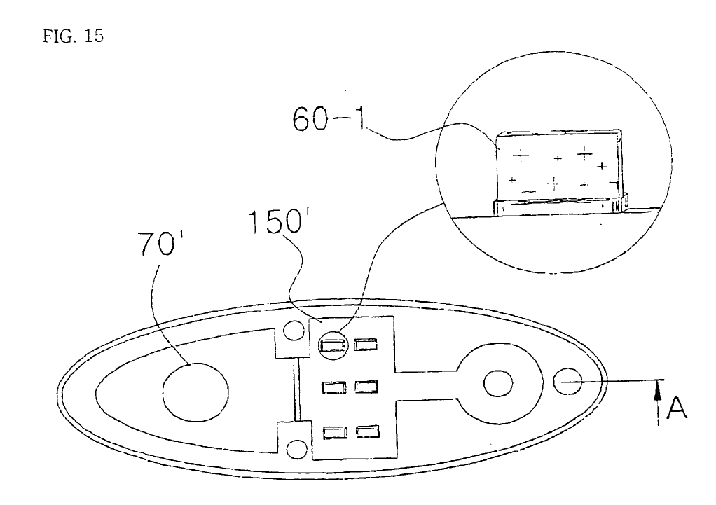

FIG. 15 illustrates another humidifying apparatus of the present invention, wherein a separate space 150′-l is formed on a central part of a body and a plurality of silver rods or silver plates 60-1 are installed inside said space 150′-1. The plurality of silver rods or silver plates are provided with an electric power source of high voltage cross-alternatively or together with heat in order to produce said silver ionized water rapidly, thereby the produced silver ionized water is vaporized by a ultrasonic wave oscillator and sprayed into air.

While the invention has been particularly shown and described with reference to preferred embodiments thereof, it will be understood by those skilled in the art that the foregoing and other chances in form and details may be made therein without departing from the spirit and scope of the invention.

### INDUSTRIAL APPLICABILITY

As described herein before, the humidifying method and apparatus of the present invention use the silver ionized water harmless to the human body and having an excellent germicidal ability itself. Therefore, the humidifying apparatus does not need to be separately sterilized and strongly inhibits the propagation of harmful virus or pathogenic bacteria. Thus, the humidifying method and apparatus of the present invention have both remarkable humidifying ability and inhibiting ability against the proliferation of harmful virus or pathogenic bacteria. Consequently, the invention can be very useful for treatment and protection of respiratory ailments caused by harmful bacteria or virus.

1. A humidifying method using silver ionized water, comprising:
producing said silver ionized water by electrolysis of a plurality of silver rods or silver plates immersed in general water, distilled water or purified water and connected to a DC or AC electric power source;
vaporizing said silver ionized water produced in said step by an ultrasonic wave oscillator, heating means or centrifugal means; and
spraying said vaporized silver ionized water into air, whereby said air is humidified and sterilized.

2. The humidifying method as claimed in claim 1, wherein said purified water is obtained from purification of said general water in an ion exchange resin system disposed in a lower end of water storing and supplying means.

3. The humidifying method as claimed in claim 1, wherein said distilled water is obtained from distillation of said general water by heating and cooling.

4. The humidifying method as claimed in claim 1, wherein said distilled water is produced by heating of said general water in distilled water generating means;
said silver ionized water is produced using said distilled water in silver ionized water generating means;
said silver ionized water is vaporized by oscillation of said ultrasonic wave oscillator;
thereby humidification by said silver ionized water is continually performed.

5. The humidifying method as claimed in claim 1, wherein said silver ionized water is rapidly produced using said general water by method for providing said plurality of silver rods or silver plates with a DC electric power source of high voltage, or
providing said plurality of silver rods, or silver plates with a DC or AC electric power source of hill voltage after heating said plurality of silver rods or silver plates by a heater connected thereto.

6. The humidifying method as in any one of claims 1 to 5, wherein said silver rod or silver plate is pure silver having a purity of at least 99.99%, and a size of silver particle electrolyzed is 0.005-0.015 μm.

7. A humidifying apparatus using silver ionized water, comprising:
a body;
a cover;
water storing and supplying means;
silver ionized water generating means;
humidification generating means comprising vaporizing means having an ultrasonic wave oscillator, heating means or centrifugal means, and spraying means having a ventilating fan; and
control means.

8. The humidifying apparatus as claimed in claim 7, further comprising an ion exchange resin system for purifying general water disposed in a lower end of said water storing and supplying means;
wherein said silver ionized water generating means are mounted in a lower end of said ion exchange resin system and comprise a plurality of silver rods or silver plates therein, said plurality of silver rods or silver plates being connected to an electric power source to be electrolyzed in water purified from said ion exchange resin system;
wherein said vaporizing means have said ultrasonic wave oscillator, whereby said silver ionized water is vaporized by said ultrasonic wave oscillator and sprayed into air.

9. The humidifying apparatus as claimed in claim 7, further comprising distilled water generating means which comprise a water reservoir, heat exchanging and vapor cooling means, a cooling plate, a cooling fan, a heater for heating said general water, a water level sensor for controlling a water level constantly, a water tube and a vapor tube,
wherein said silver ionized water Venerating means have a plurality of silver rods or silver plates therein, said plurality of silver rods or silver plates being connected to an electric power source to be electrolyzed in water distilled from said distilled water generating means, further comprising a microcomputer for providing said plurality of silver rods or silver plates with electric power source cross-alternatively;
wherein said humidification generating means further comprise a filter for removing any impurities from said silver ionized water, whereby said silver ionized water is vaporized by said ultrasonic wave oscillator and sprayed into air by said a ventilating fan.

10. The humidifying apparatus as claimed in claim 7, further comprising silver rod or silver plate fixing means having a plurality of silver rods or silver plates mounted to said water storing and supplying means;
electric power source supplying means comprising electric contacting means electrically contacted with said plurality of silver rods or silver plates and a spring mounted to said body; and
a circuit breaker for breaking an electric power source when said cover opens.

11. The humidifying apparatus as claimed in claim 10, wherein said control means comprising:
a flicker lamp mounted on a front panel of said body for displaying a electrolysis process in order while said control means provide said plurality of silver rods or silver plates with a DC or AC electric power source through said electric contacting means and silver ionization is performed by electrolysis of said plurality of silver rods or silver plates;
a sensor for preventing an electric leakage or short circuit caused by moisture; and
a circuit breaker comprising a lead switch mounted on said body and a magnetic body mounted to said cover;
whereby all parts or processes are controlled by said control means.

12. The humidifying apparatus as claimed in claim 11, further comprising a separate space formed on a central part of said body, said plurality of silver rods or silver plates being mounted in said separate space instead of being mounted in said water storing and supplying means,
wherein said plurality of silver rods or silver plates are provided with an electric power source of high voltage cross-alternatively or together with heat in order to rapidly produce said silver ionized water;
whereby said silver ionized water is vaporized by said ultrasonic wave oscillator and sprayed into air by said a ventilating fan.
